(19) United States
(12) Patent Application Publication
(10) Pub. No.: US 2024/0167173 A1
(43) Pub. Date: May 23, 2024

(54) POWER GENERATION SYSTEM AND METHOD FOR GENERATING ELECTRICITY FROM GASEOUS FLOWBACK

(71) Applicant: SAUDI ARABIAN OIL COMPANY, Dhahran (SA)

(72) Inventors: Mohammed Abdullah Bataweel, Dhahran (SA); Mustafa Alkhowaildi, Dhahran (SA); Eyad Ali Alali, Dhahran (SA); Nour Othman Baqader, Dhahran (SA); Manar Alahmari, Dhahran (SA)

(73) Assignee: SAUDI ARABIAN OIL COMPANY, Dhahran (SA)

(21) Appl. No.: 18/056,496

(22) Filed: Nov. 17, 2022

(51) Int. Cl. H02K 7/18; C01B 3/04; C01B 3/38; C01B 17/04; C07C 7/09; C25B 5/00

(52) U.S. Cl. CPC H02K 7/1815; C01B 3/04; C01B 3/382; C01B 17/0495; C07C 7/09; C25B 5/00; C01B 2203/0244; C01B 2203/06; C01B 2203/1241; C01B 2203/1258; C01B 2203/84

(57) ABSTRACT

A power generation system includes a hydrogen sulfide separator, a hydrocarbon fractionator, a hydrogen sulfide processor, a methane processor, and a hydrogen power generator. The hydrogen sulfide separator separates a gaseous flowback stream into a stream including hydrogen sulfide and a stream including hydrocarbons. The hydrocarbon fractionator fractionates hydrocarbons into methane, ethane and natural gas. The hydrogen sulfide processor converts hydrogen sulfide into hydrogen and sulfur, and the methane processor converts methane into hydrogen and carbon. The hydrogen power generator reacts hydrogen with oxygen to generate electricity. A method for generating electricity from a gaseous flowback includes separating a gaseous flowback stream into a stream including hydrogen sulfide and a stream including hydrocarbons, fractionating hydrocarbons into methane, ethane and natural gas, converting hydrogen sulfide into hydrogen and sulfur, converting methane into hydrogen and carbon, and reacting hydrogen with oxygen to generate electricity.

### BACKGROUND

Hydraulic fracturing operations in oil and gas industry generally include injecting fluid containing chemicals and other components such as proppants into a subterranean well to create new fractures or open existing fractures in the subterranean formation in order to promote the oil and gas flow for extraction. Upon completion of the fracturing operation, a high rate flowback operation may be conducted by injecting a large quantity of fluid into the subterranean well in order to remove the chemicals, proppant, polymer debris and other undesired components from the well.

The fluid produced during the flowback operation, or “flowback fluid” may include a mixture of cleanup materials and gas and liquid from the subterranean reservoir. The gaseous component of the flowback fluid or “gaseous flowback,” which may include methane and gaseous hydrocarbons, is released into atmosphere if not captured properly. A substantial amount of gaseous flowback may be discharged if the flowback operation lasts days or weeks, negatively impacting the environment. Furthermore, exploitation of a “sour reservoir,” or a reservoir containing a substantial amount of sulfur compounds, is often challenging due to the potential release of hazardous hydrogen sulfide gas during the flowback operation.

Even if the gaseous flowback is captured, the gaseous flowback is generally flared or combusted. Such flaring operation may produce carbon dioxide and other emissions associated with combustion operation, which also impacts the environment negatively. In addition, flared gaseous flowback is an unutilized energy source, and no financial gain can be obtained from the flaring operation of the gaseous flowback. Accordingly, there exists a need for continuing improvement of gaseous flowback utilization processes.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a power generation system comprising a hydrogen sulfide separator, a hydrocarbon fractionator, a hydrogen sulfide processor, a methane processor, and a hydrogen power generator.

The hydrogen sulfide separator is configured to receive a gaseous flowback stream comprising hydrocarbon and hydrogen sulfide, and separate the gaseous flowback stream into a hydrogen sulfide-containing stream comprising hydrogen sulfide and a desulfurized gaseous flowback stream comprising hydrocarbons.

The hydrocarbon fractionator is configured to receive the desulfurized gaseous flowback stream, fractionate the desulfurized gaseous flowback stream comprising hydrocarbon into a methane stream comprising methane, an ethane stream comprising ethane and a natural gas liquid stream comprising propane, butane and pentane, and fractionate the natural gas liquid stream into a propane stream comprising propane, a butane stream comprising butane and a pentane stream comprising pentane.

The hydrogen sulfide processor is configured to receive the hydrogen sulfide-containing stream, and convert hydrogen sulfide in the hydrogen sulfide-containing stream into hydrogen and sulfur to obtain a first hydrogen stream comprising hydrogen and a sulfur stream comprising sulfur.

The methane processor is configured to receive the methane stream, and convert methane in the methane stream into hydrogen and carbon to obtain a second hydrogen stream comprising hydrogen, and a carbon stream comprising carbon.

The hydrogen power generator is configured to receive at least one of the first hydrogen stream and the second hydrogen stream, and react hydrogen from at least one of the first hydrogen stream and the second hydrogen stream with oxygen to generate electricity.

In another aspect, embodiments disclosed herein relate to a method for generating electricity from a gaseous flowback. The method comprises separating a gaseous flowback stream comprising hydrogen sulfide and gaseous hydrocarbons into a hydrogen sulfide-containing stream comprising hydrogen sulfide and a desulfurized gaseous flowback stream comprising the gaseous hydrocarbons, and fractionating the desulfurized gaseous flow back stream comprising hydrocarbon. The desulfurized gaseous flow back stream is fractionated into a methane stream comprising methane, an ethane stream comprising ethane and a natural gas liquid stream comprising propane, butane and pentane. The natural gas liquid stream is fractionated into a propane stream comprising propane, a butane stream comprising butane and a pentane stream comprising pentane.

The method also comprises converting hydrogen sulfide in the hydrogen sulfide-containing stream into hydrogen and sulfur to obtain a first hydrogen stream comprising hydrogen and a sulfur stream comprising sulfur, converting methane in the methane stream into hydrogen and carbon to obtain a second hydrogen stream comprising hydrogen and a carbon stream comprising carbon, and reacting hydrogen from at least one of the first hydrogen stream and the second hydrogen stream with oxygen to generate electricity.

Other aspects and advantages of the claimed subject matter will be apparent from the following description and the appended claims.

### BRIEF DESCRIPTION OF DRAWINGS

FIG. 1 is a schematic diagram of a power generation system in accordance with one or more embodiments.

FIG. 2 is a schematic diagram of an H₂S separator in accordance with one or more embodiments.

FIG. 3 is a flow diagram of a method for generating electricity from gaseous flowback in accordance with one or more embodiments.

### DETAILED DESCRIPTION

In one aspect, embodiments disclosed herein relate to a power generation system comprising a hydrogen sulfide separator, a hydrocarbon fractionator, a hydrogen sulfide processor, a methane processor and a hydrogen power generator

In the present disclosure, a “gaseous flowback” refers to a gaseous fluid generated during the flowback operation. The gaseous flowback may include hydrogen sulfide (H₂S) and gaseous hydrocarbons. The term “gaseous hydrocarbons” refers to compounds comprising carbon and hydrogen which exist in gaseous form at room temperature, and may include methane, ethane, propane, butane and pentane, for example.

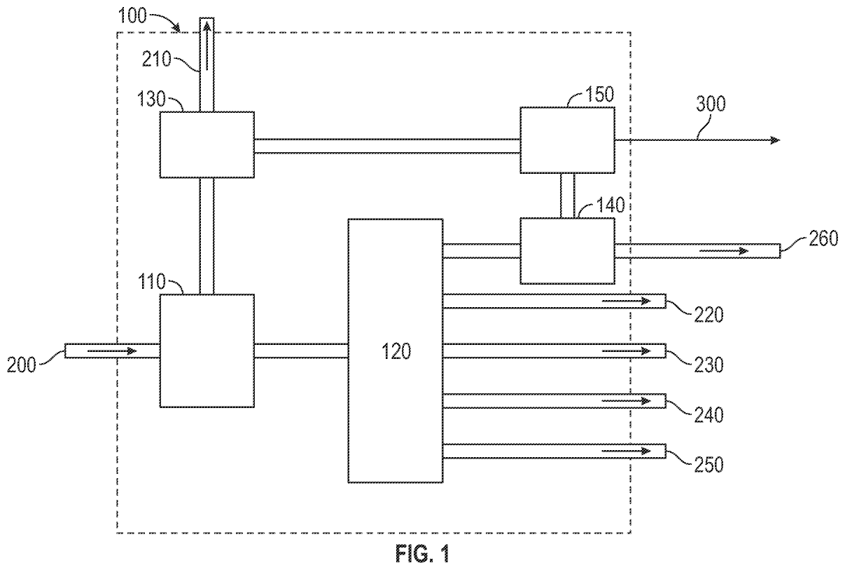

FIG. 1 is a schematic diagram of a power generation system 100. Gaseous flowback stream comprising H₂S and gaseous hydrocarbons is introduced into an H₂S separator 110 of the power generation system 100 through a gaseous flowback feed inlet 200 connected to the H₂S separator 110. The gaseous flowback feed inlet 200 may be fluidly connected to a subterranean well where the gaseous flowback stream is generated. The H₂S separator 110 separates the gaseous flowback stream into an H₂S-containing stream comprising H₂S and a desulfurized gaseous flowback stream (“desulfurized gaseous flowback”) comprising gaseous hydrocarbons. The H₂S-containing stream may also comprise water vapor and carbon dioxide. The desulfurized gaseous flowback stream may be free of H₂S gas, or may contain H₂S in an amount of 20 wt % or less of the H₂S contained in the gaseous flowback stream. The desulfurized gaseous flowback stream exits the H₂S separator 110 and enters a hydrocarbon fractionator 120 fluidly connected to the H₂S separator 110. H₂S-containing stream exits the H₂S separator 110 and enters an H₂S processor 130 fluidly connected to the H₂S separator 110.

A hydrocarbon fractionator 120 fractionates the desulfurized gaseous flowback comprising gaseous hydrocarbons into a methane stream comprising methane, an ethane stream comprising ethane and a natural gas liquid (NGL) stream comprising propane, butane and pentane. The hydrocarbon fractionator 120 further fractionates the NGL stream into a propane stream comprising propane, a butane stream comprising butane and a pentane stream comprising pentane. The methane stream exits the hydrocarbon fractionator 120 and enters a methane processor 140 fluidly connected to the hydrocarbon fractionator 120. The ethane stream, propane stream, butane stream and pentane stream exit the hydrocarbon fractionator 120 and the power generation system 100 through an ethane stream outlet 220, a propane stream outlet 230, a butane stream outlet 240 and a pentane stream outlet 250, respectively, that are separately connected to the hydrocarbon fractionator 120. In one or more embodiments, the ethane stream exits the hydrocarbon fractionator 120 and enters an ethane power generator (not shown in FIG. 1) fluidly connected to the hydrocarbon fractionator 120.

The H₂S processor 130 converts H₂S in the H₂S-containing stream into hydrogen and sulfur, which are obtained as a first hydrogen stream comprising hydrogen and a sulfur stream comprising sulfur. The first hydrogen stream exits the H₂S processor 130 and enters a hydrogen power generator 150 fluidly connected to the H₂S processor 130. The sulfur stream exits the H₂S processor 130 and the power generation system 100 through a sulfur stream outlet 210 connected to the H₂S processor 130.

The methane processor 140 converts methane into hydrogen and carbon, which are obtained as a second hydrogen stream comprising hydrogen and a carbon stream comprising carbon. The second hydrogen stream exits the methane processor 140 and enters the hydrogen power generator 150 fluidly connected to the methane processor 140. The carbon stream exits the methane processor 140 and the power generation system 100 through a carbon stream outlet 260 connected to the methane processor 140.

The hydrogen power generator 150 reacts hydrogen from at least one of the first hydrogen stream and the second hydrogen stream with oxygen to generate electricity. The generated electricity exits the power generation system 100 through a power outlet 300.

### Hydrogen Sulfide Separator

In one or more embodiments, the power generation system comprises a hydrogen sulfide (H₂S) separator (shown as 110 in FIG. 1) configured to receive a gaseous flowback stream comprising hydrocarbon and H₂S and separate the gaseous flowback stream into a H₂S-containing stream comprising H₂S and a desulfurized gaseous flowback stream comprising gaseous hydrocarbons.

In one or more embodiments, the H₂S separator in the power generation system is any gas separator capable of separating the gaseous flowback feed stream into a H₂S-containing stream comprising H₂S and a desulfurized gaseous flowback stream comprising gaseous hydrocarbons. The H₂S separator may be capable of separating at least 80 wt % of H₂S from the gaseous flowback feed stream into the H₂S-containing stream.

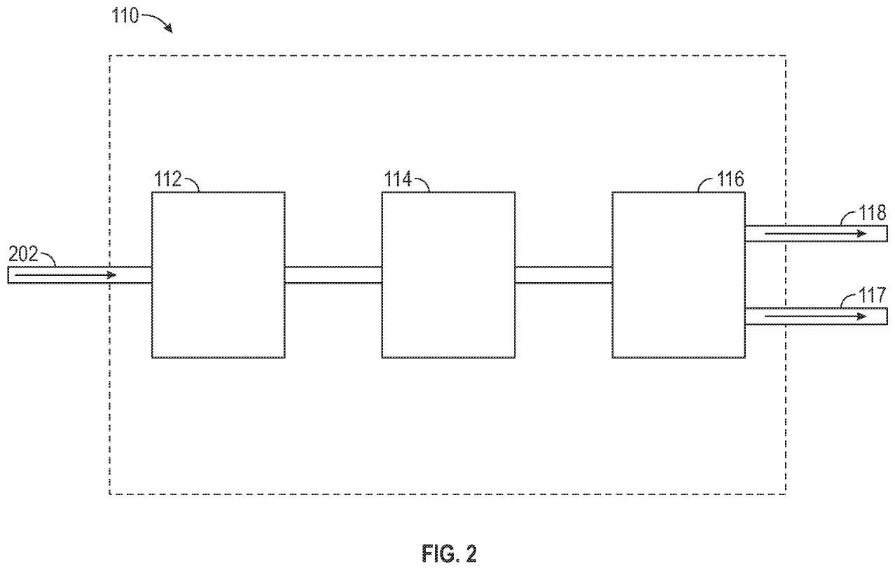

FIG. 2 is a schematic diagram of a non-limiting example of the H₂S separator. A gaseous flowback stream comprising H₂S and gaseous hydrocarbons is introduced into a H₂S separator 110 and into a filtration unit 112 through a gaseous flowback feed inlet 202 connected to the filtration unit 112 of the H₂S separator 110. The filtration unit 112 may remove undesired components contained in the gaseous flowback stream such as air-borne solid particles or debris. The gaseous flowback stream exits the filtration unit 112 and enters a heater 114 fluidly connected to the filtration unit 112. The heater 114 heats the gaseous flowback stream to a predetermined temperature. The heated gaseous flowback stream exits the heater 114 and enters a separation unit 116 fluidly connected to the heater 114. The separation unit 116 separates the gaseous flowback stream into an H₂S-containing stream comprising H₂S and a desulfurized gaseous flowback stream comprising gaseous hydrocarbons. The H₂S-containing stream exits the separation unit 116 and the H₂S separator 110 through H₂S-containing stream outlet 117 connected to the separation unit 116 and the desulfurized gaseous flowback stream exits the separation unit 116 and the H₂S separator 110 through the desulfurized gaseous flowback outlet 118 connected to the separation unit 116.

In one or more embodiments, the H₂S separator is a membrane separator comprising at least one membrane. The membrane of the membrane separator may be a commonly available membrane in the art which is capable of separating the gaseous flowback feed stream into the H₂S-containing stream and the desulfurized gaseous flowback stream. In one or more embodiments, the membrane is a membrane specifically produced to separate the gaseous flowback feed stream into the H₂S-containing stream the desulfurized gaseous flowback stream.

In case the membrane separator comprises more than one membrane, each of the membranes may have the same pore size or may have different pore sizes, and the membranes may be arranged in a parallel configuration, a series configuration, or a combination thereof. In case the membrane is arranged in a series configuration, the membranes having different pore sizes may be arranged in any order based on the requirement of the process. In one or more embodiments, the membranes may be arranged such that the membrane located at the most upstream position has the largest pore size and the downstream membranes have progressively smaller pore sizes. In one or more embodiments, the membranes may be arranged such that the membrane located at the most upstream position has the smallest pore size and the downstream membranes have progressively larger pore sizes.

In one or more embodiments, the filtration unit of the H₂S separator may be any unit capable of removing undesired components in the gaseous flowback feed stream. In one or more embodiments, the filtration unit may be a commonly available filtration system, a filter, an adsorption unit, or an absorption unit. In case the filtration unit is a filter, the filter may have a mesh size appropriate for size of the undesired components, such as solids in the gaseous flowback feed stream. The mesh size may be 100 millimeters or smaller, 50 millimeters or smaller, 10 millimeters or smaller, 5 millimeters or smaller, 1 millimeters or smaller, 100 micrometers or smaller, 50 micrometers or smaller, 10 micrometers or smaller, 5 micrometers or smaller, or 1 micrometers or smaller.

In one or more embodiments, the heater of the H₂S separator may be any commonly available heater capable of heating the gaseous flowback feed stream to an elevated temperature, such as at least 30, 40, 50, and 60° C.

A non-limiting example of the H₂S separator includes GENERON® membrane systems available from Generon Inc.

### Hydrocarbon Fractionator

In one or more embodiments, the power generation system includes a hydrocarbon fractionator (shown as 120 in FIG. 1) configured to receive the desulfurized gaseous flowback stream, fractionate the desulfurized gaseous flowback stream comprising hydrocarbons into a methane stream comprising methane, an ethane stream comprising ethane and a natural gas liquid (NGL) stream comprising propane, butane and pentane. The hydrocarbon fractionator may also be configured to fractionate the NGL stream into a propane stream comprising propane, a butane stream comprising butane and a pentane stream comprising pentane. The hydrocarbon fractionator may also be referred to as a gas separator, or a gas conditioner.

In one or more embodiments, the hydrocarbon fractionator in the power generation system is any fractionator capable of fractionating desulfurized gaseous flowback stream containing gaseous hydrocarbons into various fractionates, such as methane, ethane, and heavier fractionates including propane, butane and pentane.

In one or more embodiments, the hydrocarbon fractionator is a mechanical-refrigeration fractionator, which fractionates the desulfurized gaseous flowback stream using a cryogenic expansion process. In the mechanical refrigeration fractionator, the desulfurized gaseous flowback stream is fractionated by cooling the desulfurized gaseous flowback stream to specific temperatures to generate condensate of ethane and heavier components such as natural gas liquid (NGL) at different temperatures. Each condensate may then be retrieved separately as an ethane stream and NGL stream comprising propane, butane and pentane. A methane stream may be retrieved without condensing. The mechanical refrigeration fractionator may further fractionate the NGL stream into a propane stream, butane stream and pentane stream by heating the NGL stream to different temperatures to vaporize propane and butane, and retrieve propane and butane separately at different temperatures, and retrieve pentane, which is not vaporized.

In one or more embodiments, the hydrocarbon fractionator is an adsorption fractionator configured to separately adsorb each hydrocarbon species comprised in the desulfurized gaseous flowback stream. In one or more embodiments, the hydrocarbon fractionator is a plurality of adsorption units which may contain the same or different types of adsorbent.

In one or more embodiments, the hydrocarbon fractionator is a membrane separator. The desulfurized gaseous flowback stream may be separated by feeding the desulfurized gaseous flowback stream to the membrane separator under pressure. The separation occurs as a result of the membrane selectively rejecting heavier hydrocarbons such as NGL, and permeating lighter hydrocarbons, such as methane and ethane.

In one or more embodiments, the hydrocarbon fractionator is combinations of the fractionator/separator described above.

A non-limiting example of the hydrocarbon fractionator includes Flarecatchers™ available from Pioneer Energy, Inc.

### Hydrogen Sulfide Processor

In one or more embodiments, the power generation system comprises a hydrogen sulfide (H₂S) processor (shown as 130 in FIG. 1) configured to receive the H₂S-containing stream, convert H₂S in the H₂S-containing stream into hydrogen and sulfur to obtain a first hydrogen stream comprising hydrogen and a sulfur stream comprising sulfur from H₂S comprised in the H₂S-containing stream.

In one or more embodiments, the H₂S processor in the power generation system is any system which is capable of converting H₂S into hydrogen and sulfur.

In one or more embodiments, the H₂S processor includes a processor which converts H₂S into hydrogen and sulfur based on a Claus reaction and electrolysis process which is described in the subsequent section, and such processor is referred to as a “Claus processor” in the present disclosure. The Claus processor may comprise at least one reactor, a condenser, and an electrolysis cell. A portion of H₂S in the H₂S-containing stream may be reacted with oxygen in the reactor to convert into water and sulfur dioxide, and the produced sulfur dioxide may be further reacted in the reactor with H₂S to produce sulfur and water. A mixture gas which may comprise at least sulfur and water, and optionally H₂S and sulfur dioxide enters the condenser fluidly connected to the reactor and a portion or an entirety of sulfur may be condensed. Water in the mixture gas may then be introduced to the electrolysis cell to be converted to hydrogen and oxygen.

In one or more embodiments, the H₂S processor includes an electrolytic cell. The electrolytic cell may comprise a positive and a negative electrode where H₂S comprised in the H₂S-containing stream may be decomposed by the electrical current into hydrogen and sulfur.

In one or more embodiments, the H₂S processor includes a thermal decomposition unit. The thermal decomposition unit may comprise a furnace in which the H₂S-containing stream H₂S is heated to a specific temperature, and H₂S in the H₂S-containing stream may dissociate into hydrogen and sulfur. In one or more embodiments, H₂S-containing stream may be heated to a temperature in a range from 800 to 1200° C., or may be heated to about 1000° C. The furnace refers to any apparatus capable of receiving H₂S and heating the H₂S-containing stream to a temperature to decompose H₂S into hydrogen and sulfur, such as the temperature range as described above.

In one or more embodiments, the H₂S processor includes a catalytic processor. The catalytic processor may comprise a catalytic reactor in which the H₂S from H₂S-containing stream is converted into hydrogen and sulfur with a presence of a catalyst, such as a metal catalyst. The catalytic processor may comprise a heater such that the catalytic reactor may be heated to perform the H₂S conversion process under an elevated temperature.

In one or more embodiments, the H₂S processor includes an irradiation reactor. The irradiation reactor may comprise a vessel and at least one of microwave source and a plasma source. H₂S in H₂S-containing stream may be decomposed into hydrogen and sulfur by at least one of microwave and plasma supplied by the microwave source or the plasma source.

In one or more embodiments, the H₂S processor includes a bioreactor comprising at least one of a bioscrubber and a biofilter. A bioreactor refers to a reactor in which microorganisms, such as bacteria and fungus, are used to convert a component into different substances, such as converting H₂S into hydrogen and sulfur. The bioreactor may be an aerobic bioreactor which converts H₂S aerobically (with the presence of oxygen), or an anaerobic bioreactor, which coverts H₂S anaerobically.

In one or more embodiments, the bioreactor is a bioscrubber which may include an absorber unit, a reactor unit and an electrolysis cell. The bioscrubber may be an aerobic or anaerobic bioscrubber. An aqueous fluid and the H₂S-containing stream may be introduced into the absorber unit, and H₂S may be absorbed by the aqueous fluid to produce H₂S aqueous fluid. The H₂S aqueous fluid may then be introduced into the reactor unit where H₂S in the H₂S aqueous fluid is contacted with microorganisms contained in the reactor unit. In case of aerobic bioscrubber, the microorganism converts H₂S into sulfur and water in the presence of oxygen. Water produced in the reactor unit may be introduced to the electrolysis cell and converted into hydrogen and oxygen in the electrolysis cell. Non-limiting example of the bioscrubber may include Thiopaq® biosulfurization system available from Paques Technology B.V.

In one or more embodiments, the bioreactor is a biofilter which may include a biofilter unit comprising a biofilm. A biofilm refers to a filter in which microorganisms are imbedded securely onto the filter. The H₂S-containing stream may be introduced to the biofilter and H₂S in the H₂S-containing stream is contacted with the biofilm where transfer of H₂S from gaseous to liquid phase, and adsorption and diffusion of H₂S into the biofilm occur. The adsorbed/diffused H₂S in the biofilm is then converted to sulfur and water by the microorganisms. The biofilter may be an aerobic or anaerobic biofilter.

The biofilter may also comprise an absorber unit fluidly connected to the biofilter unit. The H₂S-containing stream may be introduced into the absorber unit and H₂S may be absorbed by the aqueous fluid to produce H₂S-containing aqueous fluid prior to being introduced into the biofilter unit.

The H₂S process may further comprise an electrolysis cell such that the water produced in the biofilm may be converted into hydrogen and oxygen in the electrolysis cell.

### Methane Processor

In one or more embodiments, the power generation system comprises a methane processor (shown as 140 in FIG. 1) configured to receive a methane stream, and convert methane in the methane stream into hydrogen and carbon to obtain a second hydrogen stream comprising hydrogen, and a carbon stream comprising carbon from methane comprised in the methane stream.

In one or more embodiments, the methane processor in the power generation system is any system which is capable of converting methane into hydrogen and carbon.

In one or more embodiments, the methane processor is a methane cracking processor. The methane cracking processor may include a methane cracking reactor in which methane is converted into hydrogen and carbon under an elevated temperature, such as at least 700° C. In one or more embodiments, the methane processor is a methane reformer comprising a H₂S reformer. H₂S from H₂S-containing stream and steam may be introduced into H₂S reformer where H₂S is reacted with steam (H₂O) to be converted into carbon dioxide and hydrogen. Non-limiting example of the methane reformer includes HY.GEN hydrogen system available from HyGear.

### Hydrogen Power Generator

In one or more embodiments, the power generation system comprises a hydrogen power generator (shown as 150 in FIG. 1) configured to receive at least one of the first hydrogen stream and the second hydrogen stream, and react hydrogen from at least one of the first hydrogen stream and the second hydrogen stream with oxygen to generate electricity.

In one or more embodiments, the hydrogen power generator in the power generation system is any system which is capable of generating electricity from hydrogen.

In one or more embodiments, the hydrogen power generator is a hydrogen engine power generator. The hydrogen engine power generator may include an electric generator, a power transmission unit, and an internal combustion engine designed to combust hydrogen to convert chemical energy into mechanical energy, and then into electrical energy. Hydrogen obtained from at least one of the H₂S processor and the methane processor is introduced into the hydrogen internal combustion engine with oxygen, and hydrogen is combusted into water under high pressure and temperature.

The internal combustion engine may be a commonly-available internal combustion engine which may comprise a combustion chamber where the fuel is combusted, and a moving component, such as piston, turbine and a rotor, which may move repeatedly as a result of the pressure generated by the combusted gas inside of the combustion chamber. The moving component may be connected to the mechanical power transmission unit which may convert the kinetic energy of the moving component into another form of kinetic energy, such as rotation of a shaft, a gear or combinations thereof.

The power transmission unit may be connected to the electric generator such that the power transmission unit provides energy by various methods, such as a rotating shaft or gears, to activate the electric generator, such as an electric motor, converting kinetic energy into electrical energy.

In one or more embodiments, the hydrogen engine power generator may include an electric generator, a power transmission unit and an external combustion engine. The external combustion engine may be a commonly available external combustion engine which may include a combustion chamber, a fluid circulation unit, and a moving component. The fluid circulation unit may be a self-contained fluid circulation system which includes a working fluid, such as water/steam. The fluid circulation unit may be configured to receive heat generated in the combustion chamber such that the working fluid in the fluid circulation unit is converted into pressurized vapor. A moving component, such as a turbine located within the fluid circulation unit, may be activated due to the flow of the pressurized vapor. Similar to the internal combustion engine, the moving component may be connected to a power transmission unit, which may be connected to an electric generator. The kinetic energy of the moving part may be converted into another kinetic energy through the power transmission unit and then into electrical energy by the electric generator.

In one or more embodiments, the hydrogen engine power generator may include a fuel cell. A fuel cell may be a commonly available fuel cell which may comprise a vessel containing electrodes. For example, the fuel cell may contain catalysts which dissociates hydrogen into hydrogen ions and react the hydrogen ions with oxygen to produce water. The dissociation of hydrogen into ions, and the reaction of hydrogen ions with oxygen result in the generation of electricity.

### Ethane Power Generator

In one or more embodiments, the power generation system includes an ethane power generator configured to receive the ethane stream and react ethane in the ethane stream with oxygen to generate electricity.

In one or more embodiments, the ethane power generator in the power generation system is any system which is capable of generating electricity from ethane.

In one or more embodiments, the ethane power generator is an engine power generator similar to the hydrogen engine power generator as previously described. The ethane power generator may be an internal combustion engine based or external combustion engine based power generator.

In one or more embodiments, the power generation system is a mobile system which can be transferred from one location to another, such as one subterranean well to another, without requiring substantial assembly and disassembly of the power generation system. Substantial assembly/disassembly refer to any assembly/disassembly of the power generation system which any of the components/units must be assembled before the system is functional, or taken down before it can be transferred. In one or more embodiments, substantial assembly and disassembly do not include simple assembly/disassembly such as connection of the components/units of the power generation system with piping, wiring and the like.

The power generation system may be made mobile by any commonly available method. In one or more embodiments, the power generation system may comprise wheels or continuous tracks, such as belts and caterpillars, which may be driven to be a self-mobile system or non-driven. In one or more embodiments, the power generation system may be placed on a mobile platform and transferred from one location to another. In one or more embodiments, the power generation system may be designed to be modular, such that each module is transferred to a desired location separately and may be made functional with a simple assembly, or be ready for transfer with a simple disassembly.

Method for Generating Electricity from a Gaseous Flowback

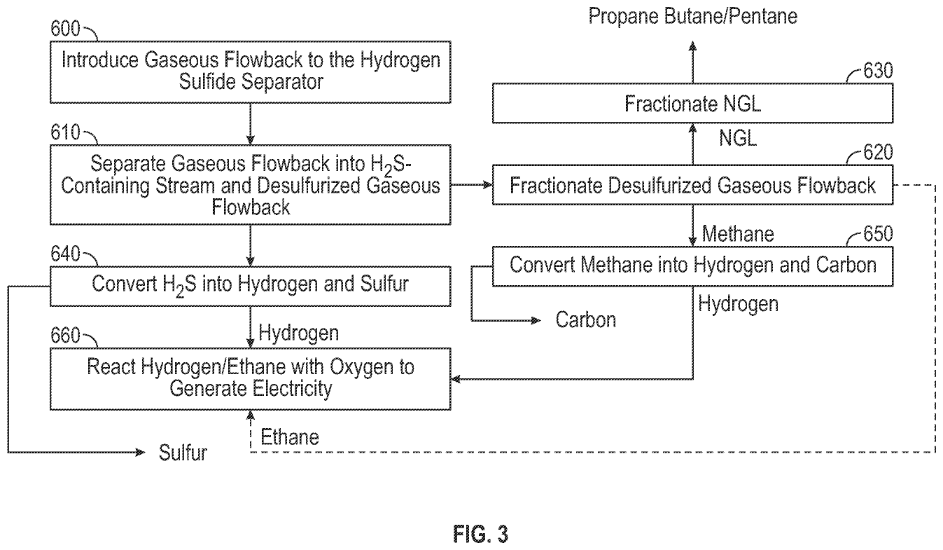

In one aspect, embodiments disclosed herein relate to a method for generating electricity from a gaseous flowback. FIG. 3 is a flow diagram of an exemplary method for generating electricity from a gaseous flowback. The method depicted in FIG. 3 may be conducted in the power generation system 100 of FIG. 1 which may contain an H₂S separator 110, a hydrocarbon fractionator 120, an H₂S processor 130, a methane processor 140 and a hydrogen power generator 150.

At 600 of FIG. 3, a gaseous flowback containing H₂S and gaseous hydrocarbons is introduced to the H₂S separator 110. The gaseous flowback may be obtained from a subterranean well.

At 610, the gaseous flowback is separated into an H₂S-containing stream comprising H₂S and a desulfurized gaseous flowback stream comprising hydrocarbons in the H₂S separator 110. The H₂S-containing stream exits the H₂S separator 110 and enters the H₂S processor 130, and the desulfurized gaseous flowback stream exits the H₂S separator 110 and enters the hydrocarbon fractionator 120.

At 620, hydrocarbons in the desulfurized gaseous flowback is fractionated in the hydrocarbon fractionator 120 into a methane stream comprising methane, an ethane stream comprising ethane, and an NGL stream comprising propane, butane and pentane.

At 630, the NGL stream is fractionated in the hydrocarbon fractionator 120 into a propane stream comprising propane, a butane stream comprising butane, and a pentane stream comprising pentane. The methane stream exits the hydrocarbon fractionator 120 and enters a methane processor 140. The ethane stream, propane stream, butane stream and propane stream exit the hydrocarbon fractionator 120.

At 640, H₂S in the H₂S-containing stream is converted into hydrogen and sulfur in the H₂S processor 130 to obtain a first hydrogen stream comprising hydrogen, and a sulfur stream comprising sulfur. The first hydrogen stream exits the H₂S processor 130 and enters the hydrogen power generator 150. The sulfur stream exits the H₂S processor 130.

At 650, methane in the methane stream is converted into hydrogen and carbon in the methane processor 140 to obtain a second hydrogen stream comprising hydrogen, and a carbon stream comprising carbon. The second hydrogen stream exits the methane processor 140 and enters the hydrogen power generator 150. The carbon stream exits the methane processor 140.

At 660, hydrogen from at least one of the first hydrogen stream and the second hydrogen stream is reacted with oxygen to generate electricity in the hydrogen power generator 150. At 660, ethane steam generated in 620 may also be reacted with oxygen to generate electricity in an ethane power generator (not shown in FIG. 1).

In one or more embodiments, the method for generating electricity from a gaseous flowback comprises separating a gaseous flowback stream comprising H₂S and gaseous hydrocarbons into a H₂S-containing stream comprising hydrogen sulfide and a desulfurized gaseous flowback stream comprising gaseous hydrocarbons. In one or more embodiments, the H₂S-containing stream may comprise water (H₂O) and carbon dioxide, in addition to H₂S.

In one or more embodiments, the separation is conducted with a commonly available method including a membrane separation process. The gaseous flowback stream may be introduced into a H₂S separator comprising at least one membrane. The membrane may be designed to be permeable to H₂S and other gases having a small molecular size, such as H₂O and carbon dioxide, and impermeable to larger molecules such as gaseous hydrocarbons. The separation may be conducted at an elevated pressure in a range from 10 psig to 2000 psig, such as a lower limit selected from any one of 10, 20, 30, 40 50 psig, to an upper limit selected from any one of 1500, 1600, 1700, 1800, 1900 and 2000 psig, where any lower limit may be selected from any upper limit.

In one or more embodiments, the gaseous flowback stream is filtered to remove undesired components prior to the separation step. The undesired components may include solid particles and debris included in the gaseous flowback stream.

In one or more embodiments, the gaseous flowback stream is heated prior to the separation step. The gaseous flowback may be heated to a temperature of at least 30, 40, 50, and 60° C. In one or more embodiments, the gaseous flowback is heated to a temperature in a range of 30 to 100° C., such as a lower limit selected from one of 30, 40, 50 and 60° C. to an upper limit selected from any one of 70, 80, 90 and 100° C., where any lower limit may be paired with any upper limit.

In one or more embodiments, the desulfurized gaseous flowback stream comprises additional components, such as impurities, in addition to gaseous hydrocarbons.

In one or more embodiments, the method for generating electricity from a gaseous flowback comprises fractionating the desulfurized gaseous flowback stream comprising gaseous hydrocarbons into a methane stream comprising methane, an ethane stream comprising ethane and a natural gas liquid stream comprising propane, butane and pentane.

In one or more embodiments, the desulfurized gaseous flowback stream is fractionated by cooling the desulfurized gaseous flowback stream to specific temperatures to generate condensate of ethane and heavier components such as natural gas liquid (NGL) comprising propane, butane and pentane at different temperatures, and retrieve each condensate separately to obtain a methane stream, ethane stream and NGL stream.

In one or more embodiments, the desulfurized gaseous flowback stream is cooled to a temperature in a range of −65 to −40° C. to condense NGL to obtain NGL stream. In order to condense NGL, the desulfurized gaseous feedback steam may be cooled to a temperature in a range from −65 to −40° C., such as a lower limit selected from any one of −65, −60 and −55° C. to an upper limit selected from any one of −50, −45 and −40° C., where any lower limit may be combined with any upper limit. In one or more embodiments, the desulfurized gaseous flowback stream is cooled further to a temperature in a range from −90 to −65° C. to condense ethane to obtain ethane stream. In order to condense ethane, the desulfurized gaseous flowback stream is cooled further to a temperature in a range from a lower limit selected from any one of −90, −85 and −80° C. to an upper limit selected from any one of −75, −70 and −65° C., where any lower limit may be paired with any upper limit. Methane may remain as a gaseous form and may be retrieved as a methane stream without being condensed. In one or more embodiments, the retrieved methane stream is a “dry” methane stream. Dry methane stream refers to a methane stream containing no moisture or have a moisture content in a range from 0 to 2 wt %, such as a lower limit selected from any one of 0, 0.001, 0.005, and 0.01 wt % to an upper limit selected from any one of 0.1, 0.5, 1, and 2 wt %, where any lower limit may be paired with any upper limit.

In one or more embodiments, the method for generating electricity from a gaseous flowback comprises fractionating the NGL stream into a propane stream comprising propane, a butane stream comprising butane and a pentane stream comprising pentane. The NGL stream obtained from the fractionation of the desulfurized gaseous flowback stream may be fractionated by adjusting the temperature of the NGL stream to a range of from −40° C. to −20° C. to vaporize propane to obtain a propane stream comprising propane. In one or more embodiments, to obtain a propane stream, the temperature of the NGL stream is adjusted to a temperature in a range from a lower limit selected from any one of −40, −37.5, −35° C. to an upper limit selected from any one of −25, −22.5 and −20° C., where any lower limit may be paired with any upper limit. The temperature of the NGL stream may be adjusted further to a temperature in a range from 0 to 20° C. to vaporize butane to obtain a butane stream comprising butane. In one or more embodiments, to obtain a butane stream, the temperature of the NGL stream is adjusted to a temperature in a range from a lower limit selected from any one of 0, 2.5 and 5° C. to an upper limit selected from any one of 15, 17.5 and 20° C., where any lower limit may be paired with any upper limit. A pentane stream comprising pentane may be retrieved as a liquid without further heating the NGL stream. The obtained propane, butane and pentane may then be consumed, stored or transported.

In one or more embodiments, the method for generating electricity from a gaseous flowback comprises converting H₂S in the H₂S-containing stream into hydrogen and sulfur to obtain a first hydrogen stream comprising hydrogen and a sulfur stream comprising sulfur. The conversion of H₂S to hydrogen and sulfur may be conducted by a variety of methods.

In one or more embodiments, the method for generating electricity from a gaseous flowback includes converting H₂S to hydrogen and sulfur by a Clause reaction/electrolysis process. The Clause reaction is a two-step reaction process represented by the following reaction steps:

H₂S+3/2O₂→H₂O+SO₂

SO₂+2H₂S→3S+2H₂O

In a Clause reaction/electrolysis process, H₂S may be introduced into a H₂S processor, such as the Clause processor previously described, and a portion of the introduced H₂S is oxidized to produce water and sulfur dioxide (SO₂). SO₂ may then be reacted with unreacted H₂S to produce sulfur and water. Water generated by Clause reaction is then converted into hydrogen and oxygen by an electrolytic process in an electrolytic cell. The conversion of water into hydrogen and oxygen is represented by the following reaction:

H₂O→H₂+1/2O₂

In one or more embodiments, the method includes converting H₂S in the H₂S-containing stream into hydrogen and sulfur by an electrolytic conversion process. The electrolytic conversion of H₂S may include introducing H₂S into an electrolysis cell containing electrolytes and applying electrical current on the electrodes of the electrolysis cell. The application of electrical current causes H₂S to dissociate into hydrogen ion and sulfur by a chemical oxidation reaction at the anode of the electrolysis cell. The reaction is represented by the following:

H₂S→2H⁺+2e⁻+1/8 S₄

Where e⁻ is an electron. The hydrogen ions produced in the above reaction convert into hydrogen gas at cathode of the electrolysis cell by the following reaction:

2H⁺+2e⁻→H₂

The above reactions require 2 moles of electrons to produce ⅛ mole of S₄ and 1 mole of hydrogen gas. Hydrogen gas may be then retrieved from the cathode of the electrolysis cell and sulfur is retrieved from the anode.

In one or more embodiments, the method for generating electricity from a gaseous flowback includes converting H₂S in the H₂S-containing stream into hydrogen and sulfur by a thermal decomposition process. H₂S may be converted to hydrogen and sulfur through the Clause reaction involving oxygen, as previously described, and dissociation of H₂O into hydrogen and oxygen. The thermal decomposition process may include introducing H₂S into an apparatus, such as a furnace, and heating H₂S to decompose into hydrogen and sulfur. The heating process may be conducted at a temperature in a range of 800 to 1200° C., such as a lower limit selected from 800, 850 and 900° C. to an upper limit selected from 1100, 1150 and 1200° C., where any lower limit may be paired with any upper limit. In one or more embodiments, the heating process is conducted at a temperature of 1000° C.

In one or more embodiments, the method for generating electricity from a gaseous flowback includes converting H₂S in the H₂S-containing stream into hydrogen and sulfur by a catalytic process. The catalytic process may include introducing H₂S into an apparatus, such as a catalytic processor comprising a catalytic reactor, and converting H₂S into hydrogen and sulfur with a catalyst, such as a heterogeneous catalyst and a metal catalyst. The metal catalyst may be used to minimize the formation of SO₂ at high temperature, and may allow the H₂S conversion at a very short contact time. The heterogeneous catalyst may allow the H₂S conversion at a low temperature. In one or more embodiments, the catalytic process may be conducted at a temperature in a range from 800 to 1200° C., such as a lower limit selected from 800, 850 and 900° C. to an upper limit selected from 1100, 1150 and 1200° C., where any lower limit may be paired with any upper limit. In one or more embodiments, the catalytic process is conducted at a temperature of about 1000° C.

In one or more embodiments, the method for generating electricity from a gaseous flowback includes converting H₂S in the H₂S-containing stream into hydrogen and sulfur by an irradiation process. The irradiation process may include introducing H₂S into an apparatus, such as an irradiation reactor, and decomposing H₂S into hydrogen and sulfur by exposing H₂S to at least one of microwave and plasma. The microwave or plasma may selectively excite the H₂S molecule for the decomposition to initiate, and the level of input energy may be controlled to initiate the decomposition. Microwave or plasma may target HSx species generated mainly from the initially excited H₂S molecule, where “x” in HSx represents a variable, and HSx includes any species generated during the irradiation process and that comprises hydrogen and sulfur. The irradiation process by plasma may be conducted at low pressure or a low H₂S concentration in order to maintain a stable plasma flow. Increase in the H₂S conversion speed may be desirable from the perspective of reduced recycling cost and decreased H₂S/H₂ separation.

In one or more embodiments, the method for generating electricity from a gaseous flowback includes converting H₂S in the H₂S-containing stream into hydrogen and sulfur by a biological decomposition process. The biological decomposition process may include introducing H₂S of H₂S-containing stream into an apparatus, such as a bioreactor previously described, and converting H₂S into hydrogen and sulfur.

As previously noted, the bioreactor may be a bioscrubber. In case a bioscrubber is used, the biological decomposition process may include absorbing H₂S into an aqueous fluid, and contacting H₂S absorbed in the aqueous fluid with microorganisms contained in the bioscrubber.

In one or more embodiments, the bioreactor may be a biofilter. In case a biofilter is used, the biological decomposition process may include contacting H₂S in the H₂S-containing stream with microorganisms, and converting H₂S into hydrogen and sulfur. The microorganisms may be securely embedded in the biofilm comprised in the biofilter. In one or more embodiments, the biological decomposition process may include absorbing H₂S into an aqueous fluid, and contacting H₂S absorbed in the aqueous fluid with microorganisms contained in the biofilm comprised in the biofilter.

In one or more embodiments, the biological decomposition process is an aerobic process. Aerobic biological decomposition process may be conducted under oxygen-limiting conditions. In such a case, oxygen may act as an electron acceptor, while H₂S may act as an electron donor, and H₂S may biologically decomposed with oxygen into water and elemental sulfur by the following reaction:

H₂S+0.5O₂→S+H₂O

The produced water may then be converted into hydrogen and oxygen by electrolytic process as previously described.

The aerobic biological decomposition process may be conducted under excess-oxygen conditions. In such a case, sulfate and hydrogen ions may be produced, which leads to the acidification of the environment surrounding the microorganisms. The reaction of such biological decomposition process may be represented as follows:

H₂S+2O₂→SO₄⁻²+2H⁺

In one or more embodiments, the biological decomposition process is an anaerobic process. In such a process, photosynthetic bacteria (phototrophs) may be used to decompose H₂S, which requires light and CO₂ but does not require oxygen for the decomposition of H₂S. The photochemical reaction to convert H₂S by phototrophs may be represented as follows:

nH₂S+n CO₂→2nS+n(CH₂O)+nH₂O

The produced water may then be converted into hydrogen and oxygen by electrolytic process as previously described.

In one or more embodiments, the method for generating electricity from a gaseous flowback comprises converting methane in the methane stream into hydrogen and carbon to obtain a second hydrogen stream comprising hydrogen and a carbon stream comprising carbon. The conversion of methane into hydrogen and carbon may be conducted by a commonly known method. In one or more embodiments, the conversion may be conducted by a methane cracking method.

The methane cracking method may include introducing methane into a methane processor, such as a methane cracking processor, and heating methane to separate hydrogen and carbon. The heating may be conducted at a temperature of at least 700, 750, 800 or 850° C. In one or more embodiments, heating of methane is conducted at a temperature in a range from 700 to 2000° C., such as a lower limit selected from any one of 700, 750, 800 and 850° C. to an upper limit selected from any one of 1850, 1900, 1950 and 2000° C., where any lower limit may be paired with any upper limit.

In one or more embodiments, the method for generating electricity from a gaseous flowback comprises reacting hydrogen from at least one of the first hydrogen stream and the second hydrogen stream with oxygen to generate electricity.

In one or more embodiments, hydrogen may be reacted with oxygen by combusting hydrogen in the presence of oxygen. The combustion of hydrogen may be conducted in a hydrogen engine power generator, for example. As described previously, a hydrogen engine power generator may comprise an electric generator, a power transmission unit and an external or internal combustion engine. Hydrogen may be combusted in the external or internal combustion engine, and the energy produced by the combustion may be converted to kinetic energy by the moving part of the internal or external engine, and transferred through the power transmission unit to activate the electric generator. The activated electric generator then converts the kinetic energy into electricity.

In one or more embodiments, hydrogen may be reacted with oxygen with a presence of a catalyst in a fuel cell. The catalyst may cause hydrogen to dissociate into hydrogen ions, and then the hydrogen ions may react with oxygen to produce water. The dissociation of hydrogen into ions, and the reaction of hydrogen ions with oxygen result in the generation of free electrons, or electricity. The electricity generated by reacting hydrogen may be used to provide power to the power generation system of the present disclosure.

In one or more embodiments, the method for generating electricity from a gaseous flowback comprises reacting ethane in the ethane stream with oxygen to generate electricity.

In one or more embodiments, ethane may be reacted with oxygen by combusting ethane in the presence of oxygen, and the combustion may be conducted in an ethane power generator such as engine power generator, as an example. The engine power generator to combust ethane may be internal or external combustion engine, and may comprise a power transmission unit and an electric generator. The electricity produced by reacting ethane may have minimum negative emission, and the electricity may be used to provide power to the power generation system of the present disclosure, or may be stored or transmitted for other uses.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. It is the express intention of the applicant not to invoke means-plus-function for any limitations of any of the claims herein, except for those in which the claim expressly uses the words ‘means for’ together with an associated function.

What is claimed is:

1. A power generation system comprising:
a hydrogen sulfide separator configured to:
receive a gaseous flowback stream comprising hydrocarbon and hydrogen sulfide; and
separate the gaseous flowback stream into a hydrogen sulfide-containing stream comprising hydrogen sulfide and a desulfurized gaseous flowback stream comprising hydrocarbons;
a hydrocarbon fractionator configured to:
receive the desulfurized gaseous flowback stream;
fractionate the desulfurized gaseous flowback stream comprising hydrocarbon into a methane stream comprising methane, an ethane stream comprising ethane and a natural gas liquid stream comprising propane, butane and pentane; and
fractionate the natural gas liquid stream into a propane stream comprising propane, a butane stream comprising butane and a pentane stream comprising pentane;
a hydrogen sulfide processor configured to:
receive the hydrogen sulfide-containing stream; and
convert hydrogen sulfide in the hydrogen sulfide-containing stream into hydrogen and sulfur to obtain a first hydrogen stream comprising hydrogen and a sulfur stream comprising sulfur;
a methane processor configured to:
receive the methane stream; and
convert methane in the methane stream into hydrogen and carbon to obtain a second hydrogen stream comprising hydrogen, and a carbon stream comprising carbon; and
a hydrogen power generator configured to:
receive at least one of the first hydrogen stream and the second hydrogen stream, and
react hydrogen from at least one of the first hydrogen stream and the second hydrogen stream with oxygen to generate electricity.

2. The power generation system of claim 1, wherein the hydrogen sulfide separator is a membrane separator.

3. The power generation system of claim 1, further comprising an ethane power generator configured to receive the ethane stream and react ethane in the ethane stream with oxygen to generate electricity.

4. The power generation system of claim 1, wherein the hydrogen sulfide processor is an electrolytic cell.

5. The power generation system of claim 1, wherein the hydrogen sulfide processor is a bioreactor.

6. The power generation system of claim 5, wherein the bioreactor is at least one of a bioscrubber and a biofilter.

7. The power generation system of claim 1, wherein the hydrogen power generator is a hydrogen engine power generator.

8. The power generation system of claim 1, wherein the power generation system is a mobile system.

9. A method for generating electricity from a gaseous flowback, the method comprising:
separating a gaseous flowback stream comprising hydrogen sulfide and gaseous hydrocarbons into a hydrogen sulfide-containing stream comprising hydrogen sulfide and a desulfurized gaseous flowback stream comprising the gaseous hydrocarbons;
fractionating the desulfurized gaseous flowback stream comprising hydrocarbon into a methane stream comprising methane, an ethane stream comprising ethane and a natural gas liquid stream comprising propane, butane and pentane;
fractionating the natural gas liquid stream into a propane stream comprising propane, a butane stream comprising butane and a pentane stream comprising pentane;
converting hydrogen sulfide in the hydrogen sulfide-containing stream into hydrogen and sulfur to obtain a first hydrogen stream comprising hydrogen and a sulfur stream comprising sulfur;
converting methane in the methane stream into hydrogen and carbon to obtain a second hydrogen stream comprising hydrogen and a carbon stream comprising carbon; and
reacting hydrogen from at least one of the first hydrogen stream and the second hydrogen stream with oxygen to generate electricity.

10. The method of claim 9, wherein the separating is conducted by at least one membrane.

11. The method of claim 9, wherein the hydrogen sulfide-containing stream comprises at least 80 wt % of hydrogen sulfide comprised in the gaseous flowback stream.

12. The method of claim 9, wherein the hydrogen sulfide-containing stream further comprises water and carbon dioxide.

13. The method of claim 9, wherein the methane stream is a dry methane stream.

14. The method of claim 9, wherein the converting hydrogen sulfide is conducted by at least one process selected from the group consisting of a Clause reaction/electrolysis process, an electrolytic conversion process, a thermal decomposition process, a catalytic process, an irradiation process, and a biological decomposition process.

15. The method of claim 14, wherein the biological decomposition process is conducted in at least one of a bioscrubber and a biofilter.

16. The method of claim 14, wherein the biological decomposition process is aerobic.

17. The method of claim 14, wherein the biological decomposition process is anaerobic.

18. The method of claim 9, wherein the fractionating the desulfurized gaseous hydrocarbons is conducted at a temperature in a range from −90° C. to −40° C.

19. The method of claim 9, wherein the converting methane is conducted by a methane cracking process at a temperature of at least 700° C.

20. The method of claim 9, further comprising reacting ethane in the ethane stream with oxygen to generate electricity.
